(19) United States
(12) Patent Application Publication
(10) Pub. No.: US 2024/0202595 A1
(43) Pub. Date: Jun. 20, 2024

(54) MACHINE LEARNING ENABLED EXTRACTION OF UNSTRUCTURED CLINICAL DATA

(71) Applicant: CareFusion 303, Inc., San Diego, CA (US)

(72) Inventors: Abhikesh Nag, San Diego, CA (US); Christopher Hull, San Diego, CA (US); Gautam Sampath, San Diego, CA (US)

(21) Appl. No.: 18/540,101

(22) Filed: Dec. 14, 2023

Related U.S. Application Data
(60) Provisional application No. 63/387,668, filed on Dec. 15, 2022.

(51) Int. Cl. G06N 20/00

(52) U.S. Cl. CPC G06N 20/00

(57) ABSTRACT

A method may include receiving, from one or more data systems, a message including unstructured clinical data. A machine learning model may be applied to identify a first entity and a second entity present in the unstructured clinical data. The first entity and the second entity may occupy a same row or successive rows in a same column of the unstructured clinical data. The machine learning model may be trained to determine, based at least on the unstructured clinical data including the first entity, that the second entity is a most likely entity occupying a next position in the same row or a next row in the same column of the unstructured clinical data. Clinically significant data may be extracted from the structured clinical data. At least one medical device may be controlled, based at least on the clinically significant data, to perform one or more tasks.

### CROSS-REFERENCE TO RELATED APPLICATION

This application claims priority to U.S. Provisional Application No. 63/387,668, entitled “MACHINE LEARNING ENABLED EXTRACTION OF UNSTRUCTURED CLINICAL DATA” and filed on Dec. 15, 2022, the disclosure of which is incorporated herein by reference in its entirety.

### TECHNICAL FIELD

The subject matter described herein relates generally to machine learning and more specifically to a machine learning based technique for extracting unstructured clinical data.

### BACKGROUND

Laboratory automation is a multi-disciplinary effort to research and develop technologies to optimize the speed, efficiency, and outcome of laboratory workflows. For example, a laboratory information system (LIS) may include hardware and software configured to provide support for laboratory activities such as inoculation, incubation, plate imaging, culture reading, result reporting, and/or the like. The laboratory information system may record, analyze, store, and share data generated by various laboratory activities. In doing so, the laboratory information system may aim to deliver timely, accurate, and relevant information, whether in clinical settings where the focus tends to be on patient-specific specimen or in non-clinical settings such as research laboratories and/or the like.

### SUMMARY

Systems, methods, and articles of manufacture, including computer program products, are provided for a machine learning based extraction engine. In some example embodiments, there is provided a system that includes at least one processor and at least one memory. The at least one memory may include instructions that cause operations when executed by the at least one processor. The operations may include: receiving, from one or more data systems, a message including unstructured clinical data; applying a machine learning model to identify a first entity and a second entity present in the unstructured clinical data, the first entity and the second entity occupying a same row of the unstructured clinical data or successive rows in a same column of the unstructured clinical data, the machine learning model being trained to determine, based at least on the unstructured clinical data including the first entity, that the second entity is a most likely entity occupying a next position in the same row or a next row in the same column of the unstructured clinical data; extracting, from the structured clinical data, a clinically significant data; and controlling, based at least on the clinically significant data, at least one medical device to perform one or more tasks.

In another aspect, there is provided a method for a machine learning based extraction engine. The method may include: receiving, from one or more data systems, a message including unstructured clinical data; applying a machine learning model to identify a first entity and a second entity present in the unstructured clinical data, the first entity and the second entity occupying a same row of the unstructured clinical data or successive rows in a same column of the unstructured clinical data, the machine learning model being trained to determine, based at least on the unstructured clinical data including the first entity, that the second entity is a most likely entity occupying a next position in the same row or a next row in the same column of the unstructured clinical data; extracting, from the structured clinical data, a clinically significant data; and controlling, based at least on the clinically significant data, at least one medical device to perform one or more tasks.

In another aspect, there is provided a computer program product that includes a non-transitory computer readable medium storing instructions. The instructions may cause operations when executed by at least one data processor. The operations may include: receiving, from one or more data systems, a message including unstructured clinical data; applying a machine learning model to identify a first entity and a second entity present in the unstructured clinical data, the first entity and the second entity occupying a same row of the unstructured clinical data or successive rows in a same column of the unstructured clinical data, the machine learning model being trained to determine, based at least on the unstructured clinical data including the first entity, that the second entity is a most likely entity occupying a next position in the same row or a next row in the same column of the unstructured clinical data; extracting, from the structured clinical data, a clinically significant data; and controlling, based at least on the clinically significant data, at least one medical device to perform one or more tasks.

In some variations of the methods, systems, and computer program products, one or more of the following features can optionally be included in any feasible combination.

In some variations, the machine learning model is trained to identify the second entity included in the unstructured clinical data by at least: determining, based at least on the unstructured clinical data including the first entity, a first probability that the second entity is the most likely entity occupying the next position in the same row or the next row in the same column of the unstructured clinical data, determining, based at least on the unstructured clinical data including the first entity, a second probability that a third entity is the most likely entity occupying the next position in the same row or the next row in the same column of the unstructured clinical data, identifying the second entity based at least on a comparison of the first probability and the second probability.

In some variations, an entity graph including the first entity and the second entity may be generated based at least on an alignment between the first entity and the second entity in the unstructured clinical data.

In some variations, a horizontal chain of the entity graph may be generated, based at least on a horizontal alignment between the first entity and the second entity, to include the first entity and the second entity such that the first entity and the second entity occupy a single row.

In some variations, a vertical chain of the entity graph may be generated, based at least on a vertical alignment between the first entity and the second entity, to include the first entity and the second entity such that the first entity and the second entity occupy successive rows in a single column.

In some variations, the machine learning model may identify the first entity and the second entity by at least assigning, to each of the first entity and the second entity, a corresponding entity marker.

In some variations, the at least one medical device may be controlled based on at least one of a header, an organism name, a drug name, a concentration value, and an interpretation value comprising the first entity and/or the second entity.

In some variations, the machine learning model may be trained based at least on training data including annotated unstructured clinical data to learn one or more entity patterns that are present in the annotated unstructured clinical data. The annotated unstructured clinical data may include a plurality of entities annotated with a corresponding ground truth entity marker.

In some variations, the one or more entity patterns may include a co-occurrence of the first entity and the second entity in a single row or in successive rows of a single column.

In some variations, the one or more tasks may include: identifying, based at least on the clinically significant data, a stage of a clinical workflow associated with the one or more messages, determining, based at least on a timestamp associated with the one or more messages, a quantity of time between two or more successive stages of the clinical workflow, and in response to the quantity of time between the two or more successive stages of the clinical workflow exceeding a threshold value, determining one or more corrective configurations for the at least one medical device.

In some variations, the one or more corrective actions may include modifying a scheduling of one or more activities associated with the clinical workflow and/or adjusting an allocation of resources associated with the one or more activities.

In some variations, the identifying of the stage of the clinical workflow may include identifying the stage of a microbial testing workflow and/or a virology assay.

In some variations, the identifying the stage of the clinical workflow may include identifying a start of a culturing process for a microbe, a gram positive or gram negative identification for the microbe, a species and/or organism identification for the microbe, or an antimicrobial susceptibility of the microbe.

In some variations, the one or more tasks may include determining, based at least on the clinically significant data, an allocation of resources at the one or more data systems.

In some variations, the allocation of resources may include: determining, based at least on the clinically significant data, a subsequent stage of a clinical workflow and a time for the subsequent stage of the clinical workflow, and scheduling, in accordance with the time of the subsequent stage of the clinical workflow, a quantity of resources required for the subsequent stage of the clinical workflow.

In some variations, one or more second machine learning models may be applied to extract, from the structured clinical data, the clinically significant data.

In some variations, the one or more second machine learning models may be trained to identify and tag the clinically significant data included in the structured clinical data. The one or more second machine learning models may be further trained to determine that the message associated with the structured clinical data is actionable in response to more than a threshold portion of the structured clinical data being tagged as clinically significant.

In some variations, the one or more second machine learning models may be further trained to determine, based at least on a sequence of messages including the message, whether the sequence of messages is actionable.

In some variations, the controlling of the at least one medical device may include transmitting, to the at least one medical device, one or more messages to adjust an operational state and/or a functional element of the at least one medical device.

In some variations, the one or more messages may include one or more instructions, which when executed by a processor associated with the at least one medical device, adjust the operational state and/or the functional element of the at least one medical device.

In some variations, the one or more messages may include one or more values, which when applied at the at least one medical device, adjust the operational state and/or the functional element of the at least one medical device.

In some variations, the at least one medical device may be an infusion pump including a pumping functional element. The method may include generating the one or more messages to adjust a pumping rate of the pumping functional element.

In some variations, the at least one medical device may be a dispensing cabinet or a wasting station including one or more receptacles. The method may include generating the one or more messages to control access to the one or more receptacles.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to machine learning enabled extraction of unstructured clinical data, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a clinical management system, in accordance with some example embodiments;

FIG. 2 depicts a schematic diagram illustrating an example of a dataflow within a clinical management system, in accordance with some example embodiments;

FIG. 3A depicts a schematic diagram illustrating an example of entity marking, in accordance with some example embodiments;

FIG. 3B depicts a schematic diagram illustrating an example of entity pattern recognition, in accordance with some example embodiments;

FIG. 4A depicts a schematic diagram illustrating an example of an entity graph, in accordance with some example embodiments;

FIG. 4B depicts a schematic diagram illustrating a horizontal chain and a vertical chain in an example of an entity graph, in accordance with some example embodiments;

FIG. 4C depicts an example of a coordinate document, in accordance with some example embodiments;

FIG. 5 depicts a screenshot illustrating an example of a user interface for annotating unstructured clinical data, in accordance with some example embodiments;

FIG. 6A depicts a flowchart illustrating an example of a process for updating a machine learning model trained to extract unstructured clinical data, in accordance with some example embodiments;

FIG. 6B depicts a flowchart illustrating an example of a process for machine learning enabled extraction of unstructured clinical data, in accordance with some example embodiments; and

FIG. 7 depicts a block diagram illustrating a computing system, in accordance with some example embodiments.

When practical, similar reference numbers denote similar structures, features, or elements.

### DETAILED DESCRIPTION

A laboratory information system (LIS) may be deployed in clinical and non-clinical settings to support various laboratory activities such as inoculation, incubation, plate imaging, culture reading, result reporting, and/or the like. The laboratory information system may record, analyze, store, and share data arising from such laboratories activities. For example, the laboratory information system may generate a variety of messages at each stage of a microbial test, virology assay, and/or the like. Some messages may convey clinically significant data and are therefore actionable messages associated with additional tasks while others, such as transitional status messages and/or the like, are non-actionable messages. Moreover, these messages may include unstructured clinical data that varies across different medical devices, clinical facilities, laboratory information systems, and automation platforms. Inconsistency in the content and format of the messages may thwart efforts to establish a uniform data interface across devices, facilities, and platforms. This lack of interoperability may compromise the speed, efficiency, and outcome of various clinical workflows. Downstream decisions, such as those involving workflow statistics or strategic resource allocation or therapeutic administration devices, may suffer in the absence of timely, accurate, and relevant information from the laboratory information systems.

In some example embodiments, a message exchange may receive, from a variety of sources across different medical devices, clinical facilities, and automation platforms, messages containing unstructured clinical data. For example, the message exchange may receive messages from one or more laboratory information systems (LISs) engaged in a workflow such as microbial testing, virology assay, and/or the like. The message exchange may include an extraction engine configured to parse the unstructured clinical data contained in each message. For instance, in some cases, the extraction engine may apply one or more machine learning models trained to recognize the entities and the patterns formed therefrom (e.g., combinations of entities, sequences of entities, and/or the like) present in the unstructured clinical data. Examples of the entities that may be present in the unstructured clinical data include headers, organism names, drug names, concentration values (e.g., minimum inhibitory concentration (MIC) values), interpretation values, and/or the like. In some cases, the extraction engine may generate structured clinical data by at least populating a data structure (e.g., a vector, a matrix, a table, and/or the like) with the values of the entities extracted from the unstructured clinical data. Unlike unstructured clinical data, the structured clinical data generated by the extraction engine may be more suitable for various downstream tasks including but not limited to controlling medical devices.

In some example embodiments, an analysis engine may perform one or more downstream analytical tasks based on the structured clinical data received from the message exchange. For example, in some cases, the analysis engine may apply one or more machine learning models trained to identify actionable messages and extract clinically significant data to enable clinical decisions such as the collection of workflow statistics, resource allocation, and/or the like. For the microbial testing workflow, for example, the analysis engine may apply the one or more machine learning models to identify actionable messages indicating a start of a culturing process for a microbe, a gram positive or gram negative identification for the microbe, a species and/or organism identification for the microbe, an antimicrobial susceptibility of the microbe, and/or the like. Moreover, the machine learning model may identify, within the structured clinical data associated with each actionable message, clinically significant data to enable downstream clinical actions and decisions. For instance, the output of the machine learning model may include the identification of cultural isolates and the results of antimicrobial susceptibility tests (AST), which may form the basis of downstream decisions in resource allocation, treatment, and/or the like. The availability of timely, accurate, and relevant information from the laboratory information system may expedite the clinical actions needed to render effective antimicrobial treatment, reduce antimicrobial resistance risk, and improve patient outcome. The information provided by the features described may further reduce the resources used by a clinical device to administer a therapy based on LIS data because the data will be in more standardized and in a more machine-consumable format. For example, if an infusion pump receives information regarding a patient's antimicrobial resistance, the time to receive, process, and take action based on the information is reduced when the information is generated using the described features because the device may forego resource intensive analysis of unstructured data instead relying on the standard, single format, output from the features described. In some implementations, the device may more quickly identify treatment that should be stopped or changed based on LIS information. The resource savings ensure device resources such as battery, processor time, or network bandwidth are available for more critical functions.

The features described further facilitate safe and efficient integration of additional LIS sources. Because the features allow unstructured data to be transformed into an actionable data set, the system can respond to new, previously unknown LIS formats with minimal disruption to downstream, consuming devices. In this way, adding a new LIS may involve training a message exchange to process the new LIS's data, but to any of peer device within the broader facility, the message can be processed in the same fashion as messages based on data from a legacy LIS.

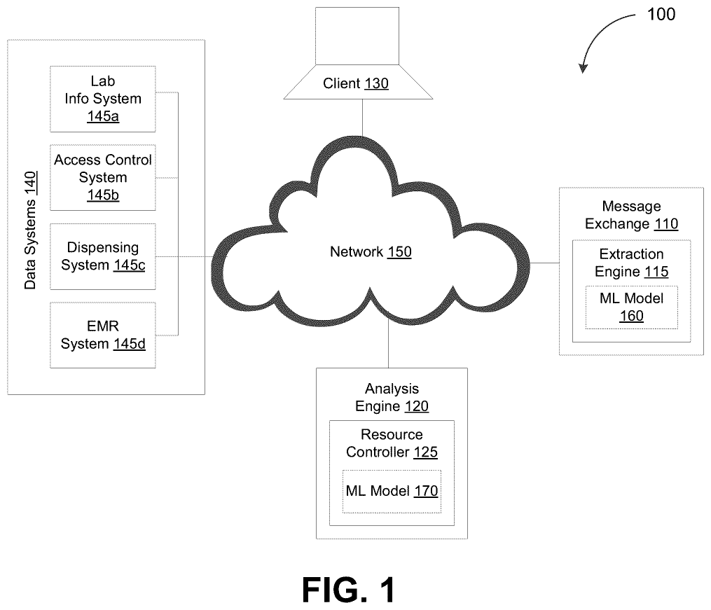

FIG. 1 depicts a system diagram illustrating an example of a clinical management system 100, in accordance with some example embodiments. Referring to FIG. 1, the clinical management system 100 may include a message exchange 110 including an extraction engine 115, an analysis engine 120 including a resource controller 125, a client 130, and one or more data systems 140. As FIG. 1 shows, the message exchange 110, the analysis engine 120, the client 130, and the one or more data systems 140 may be communicatively coupled via a network 150. The client 130 may be a processor-based device including, for example, a smartphone, a tablet computer, a wearable apparatus, a desktop computer, a laptop computer, a workstation, and/or the like. Meanwhile, the network 150 may be any wired and/or wireless network including, for example, a public land mobile network (PLMN), a local area network (LAN), a virtual local area network (VLAN), a wide area network (WAN), the Internet, and/or the like.

The message exchange 110 and the analysis engine 120 may be accessible to the client 130 as a cloud-based service (e.g., a software-as-a-service (SaaS) and/or the like). Alternatively, the message exchange 110 and/or the analysis engine 120 may be at least partially embedded and/or implemented within the one or more data systems such as, for example, at a laboratory information system (LIS) 145a, an access control system 145b, a dispensing system 145c, an electronic medical record (EMR) system 145d, and/or the like. That is, the message exchange 110 and/or the analysis engine 120 may be at least partially embedded and/or implemented within a medical device such as, for example, a dispensing cabinet, an infusion pump, a wasting station, and/or the like. For example, in some example embodiments, the message exchange 110 may be a centralized, cloud-based service whereas the analysis engine 120 may be deployed across the data systems 140. Accordingly, at least some functionalities of the message exchange 110 and/or the analysis engine 120 may be accessed locally at the one or more data systems 140. Moreover, the message exchange 110 and/or the analysis engine 120 may be updated and/or configured as part of servicing and/or updating the corresponding data systems 140.

The message exchange 110 may receive messages generated by the one or more data systems 140 including, for example, the laboratory information system 145a, the access control system 145b, the dispensing system 145c, the electronic medical record system 145d, and/or the like. Some messages may convey clinically significant data and are therefore actionable messages associated with additional tasks while others, such as transitional status messages and/or the like, are non-actionable messages. Moreover, these messages may include unstructured clinical data whose format and/or content may vary across different medical devices, clinical facilities, and automation platforms.

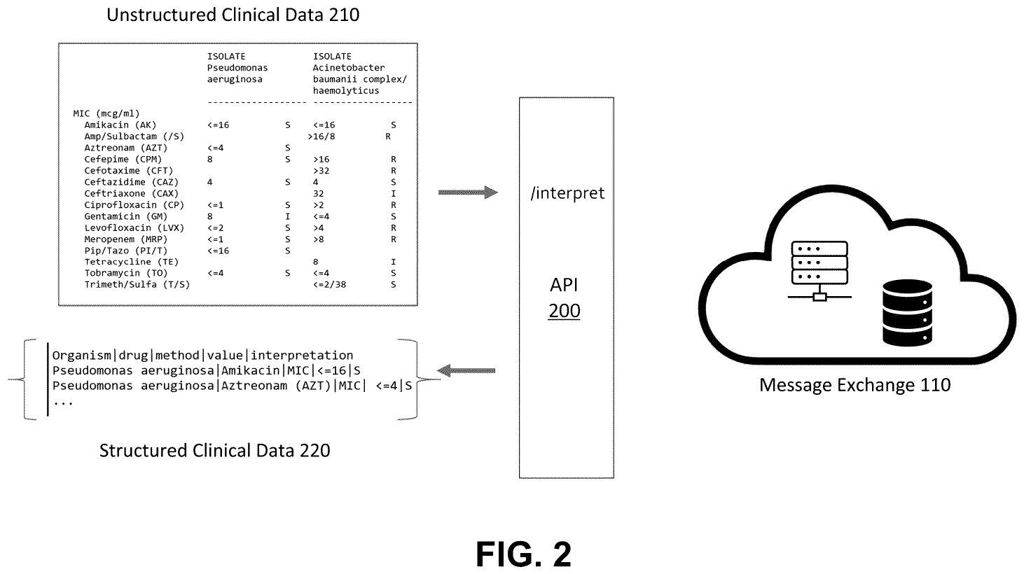

Accordingly, in some example embodiments, the extraction engine 115 at the message exchange 110 may parse the unstructured clinical data contained in each message. For example, in the example dataflow shown in FIG. 2, the message exchange 110 may receive, via an application programming interface 200, one or more requests (e.g., /interpret) containing unstructured clinical data 210. In some cases, the unstructured clinical data 210 may include text strings that lack consistency in content and format. The extraction engine 115 may respond to the one or more requests by generating structured clinical data 220 corresponding to the unstructured clinical data 210. For example, in some cases, the extraction engine 115 may apply a machine learning model 160 trained to recognize the various entities and the patterns formed therefrom (e.g., combinations of entities, sequences of entities, and/or the like) that are present in the unstructured clinical data 210. Examples of the entities that may be present in the unstructured clinical data 210 include headers, organism names, drug names, concentration values (e.g., minimum inhibitory concentration (MIC) values), interpretation values, and/or the like. Moreover, to generate the structured clinical data 220, the extraction engine 115 populate a data structure (e.g., a vector, a matrix, a table, and/or the like) with the values of the entities extracted from the unstructured clinical data 210.

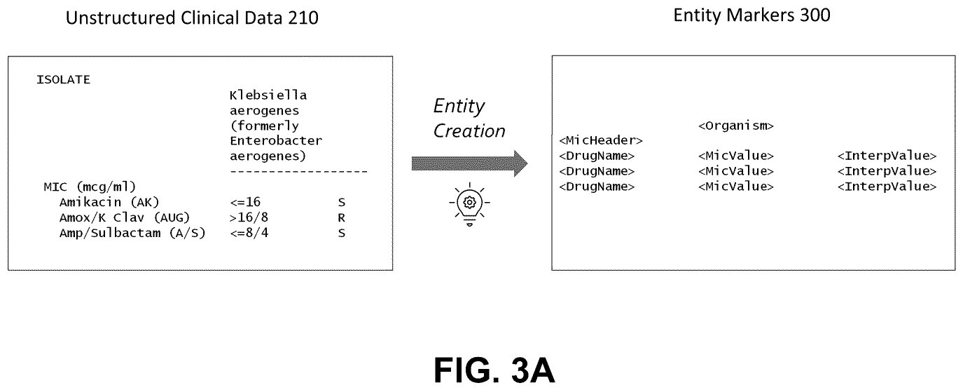

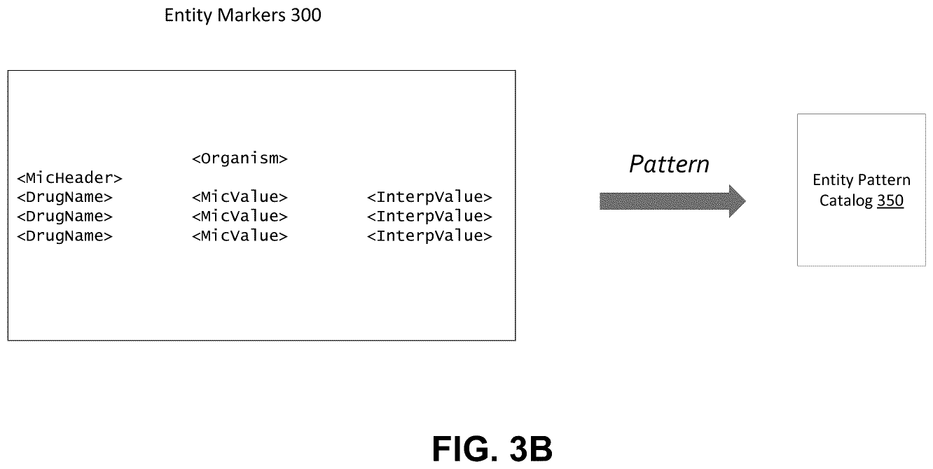

To further illustrate, FIG. 3A depicts an example of entity marking in which the machine learning model 160 is applied to identify, within the unstructured clinical data 210, one or more entities such as headers, organism names, drug names, concentration values (e.g., minimum inhibitory concentration (MIC) values), interpretation values, and/or the like. In the example shown in FIG. 3A, the machine learning model 160 may identify each entity present in the unstructured clinical data 210 by at least assigning, to each entity, a corresponding entity marker 300. For example, in some cases, the entity marker 300 assigned to an entity in the unstructured clinical data 210 may identify the entity as, for example, a header, an organism name, a drug name, a concentration value (e.g., a minimum inhibitory concentration (MIC) value), an interpretation value, and/or the like.

FIG. 3B depicts a schematic diagram illustrating an example of entity pattern recognition, in accordance with some example embodiments. As noted, the machine learning model 160 may be trained to recognize, based at least on the entity markers 300, the patterns that are present in the entities identified within the unstructured clinical data 210. In this context, two or more entities present in the unstructured clinical data 210 may form a pattern if the two or more entities are known to co-occur (e.g., in the training data used to train the machine learning model 160) at a frequency that satisfy one or more thresholds. For example, a first entity and a second entity may form a pattern if the frequency at which the first entity appear adjacent to the second entity exceeds a threshold value. In some cases, this pattern may include a co-occurrence of the first entity and the second entity regardless of the order in which the two entities appear in the unstructured clinical data 210. In other cases, this pattern may include the first entity and the second entity appearing a specific order in the unstructured clinical data 210. That is, the machine learning model 160 may be trained to recognize a certain sequence of entities that includes, for example, the first entity followed by the second entity. For instance, the pattern of entities may include the first entity appearing before the second entity in a same row the unstructured clinical data 210 or the first entity appearing in one row the unstructured clinical data 210 and the second entity appearing in a subsequent row of the unstructured clinical data 210. In the example shown in FIG. 3B, various entity patterns that are present in unstructured clinical data, such as the unstructured clinical data 210, may be included an entity pattern catalog 350. Moreover, as will be described in more detail below, the machine learning model 160 may be trained, based on at least on the entity pattern catalog 350, to learn the various entity patterns that may be present in unstructured clinical data such as the unstructured clinical data 210.

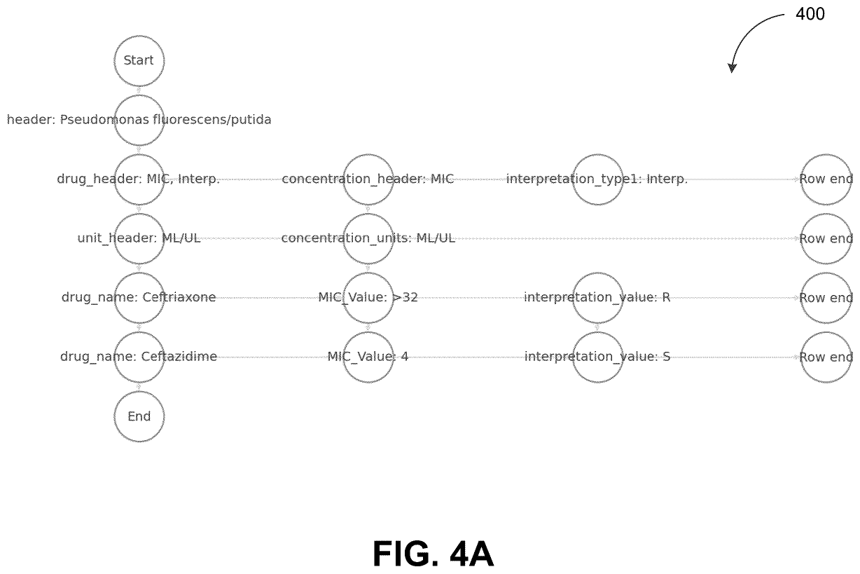

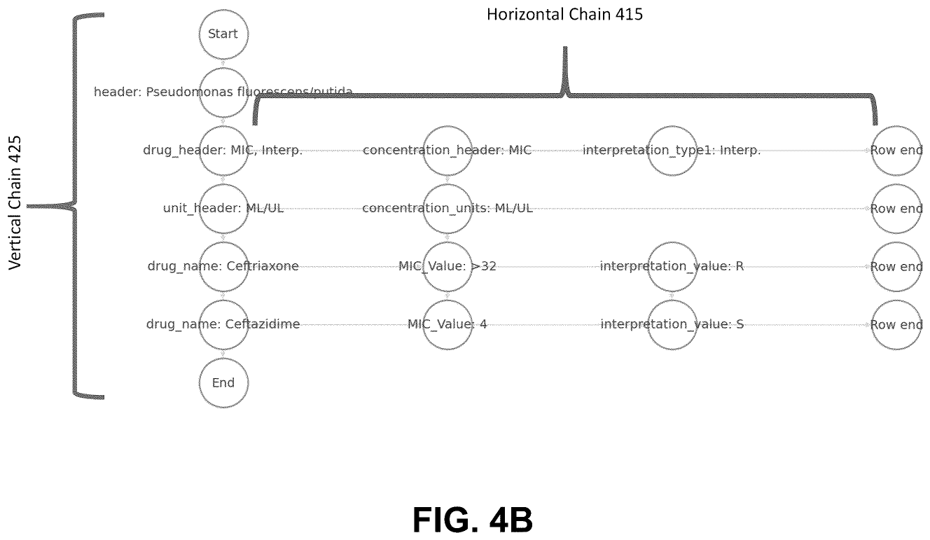

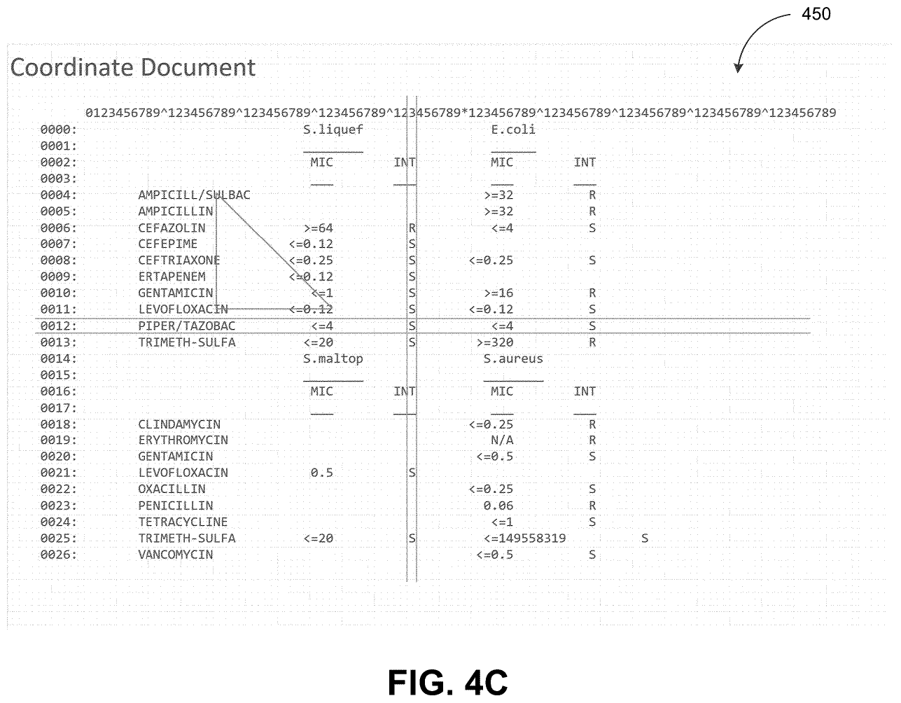

As noted, in some cases, the machine learning model 160 may be trained to recognize, within the unstructured clinical data 210, certain entity patterns in which two or more entities appear in a specific sequence in a same row of the unstructured clinical data 210 and/or across successive rows of the unstructured clinical data 210. In some cases, the pattern of entities included in the unstructured clinical data 210 may form an entity graph in which sequences of entities form horizontal chains as well as vertical chains. In this context, a horizontal chain of entities may include two or more entities that occupy a same row of the unstructured clinical data 210 while a vertical chain of entities may include two or more entities that occupy a same column of the unstructured clinical data 210. To further illustrate, FIG. 4A depicts an example of an entity graph 400 in which sequences of entities form horizontal chains as well as vertical chains. FIG. 4B depicts an example of a horizontal chain 415 including the entities drug_header, concentration_header, interpretation_type, and row end. Furthermore, FIG. 4B depicts an example of a vertical chain 425 including the entities header, drug_header, unit_header, drug_name, and drug_name. FIG. 4C depicts an example of a coordinate document 450 in which entities included in the unstructured clinical data 210 are aligned to form corresponding vertical chains and horizontal chains of entities. In some cases, the coordinate document 450 may define one or more vertical chains and horizontal chains based at least on a relative location of the entities included in the unstructured clinical data 210. For example, the coordinate document 450 may define a horizontal chain in an entity graph to include a first entity and a second entity based at least on the horizontal alignment between the first entity and the second entity. Alternatively and/or additionally, the coordinate document 450 may define a vertical chain in the entity graph to include the first entity and the second entity based at least on the vertical alignment between the first entity and the second entity.

In some example embodiments, the machine learning model 160 may be a conditional random fields (CRF) model that has been trained, for example, based on the entity pattern catalog 350, to learn the various entity patterns that may be present in unstructured clinical data such as the unstructured clinical data 210. In some cases, the conditional random fields (CRF) model may be defined on observations X and random variables Y. In this context, the observations X may correspond to an entity identified within a linear chain (e.g., horizontal chain or vertical chain) in the unstructured clinical data 210 whereas random variables Y may correspond to a next entity in the same linear chain. The definition of the conditional random fields (CRF) model may include a graph G(V, E) corresponding, for example, to the example of the entity graph 400 shown in FIG. 4A. The random variables Y may be indexed by the vertices V of the graph G such that Y=(Yᵥ)_{v∈V}. The tuple (X, Y) may be a conditional random field when each random variable Yᵥ, conditioned on X, obeys the Marko property with respect to the graph G, meaning that the probability P of Yᵥ being a particular entity may be dependent only on the entities corresponding to its neighboring vertices in the graph G. Accordingly, P(Yᵥ|X, {Y_{w}: w≠v})=P(Yᵥ|X, {Y_{w}: w˜v}), where the notation w˜v indicates that the corresponding vertices are neighbors in the graph G.

Accordingly, when implemented as a conditional random fields (CRF) model, the machine learning model 160 may be assign, based at least on the entity patterns learned through training, entity markers to each entity present in the unstructured clinical data 210. For example, the machine learning model 160 may be trained to learn an entity pattern in which the first entity and the second entity form a horizontal chain (e.g., the first entity appears before the second entity in a same row). Accordingly, upon identifying the first entity in the unstructured clinical data 210, the machine learning model 160 may determine that the second entity is the most likely entity occupying the next position in the same row of the unstructured clinical data 210. Alternatively and/or additionally, the machine learning model 160 may be trained to learn an entity pattern in which the first entity and the second entity form a vertical chain (e.g., the first entity and the second entity appearing in successive rows of a same column). As such, upon identifying the first entity in one row of the unstructured clinical data 210, the machine learning model 160 may determine that the second entity is the most likely entity occupying a subsequent row in the same column of the unstructured clinical data 210.

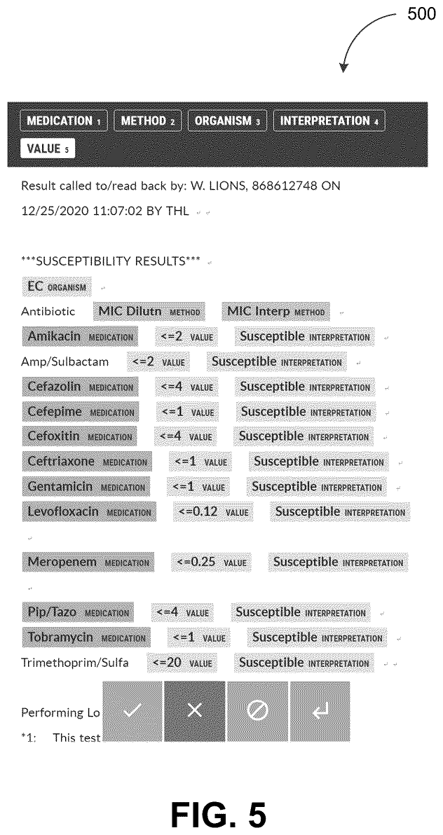

In some example embodiments, the machine learning model 160 may be trained based on annotated training data such as, for example, an initial set of messages that have been annotated with labels (e.g., ground truth entity marker labels) of at least a portion of the entities contained therein. In some cases, the labels may be applied based on one or more user inputs (e.g., expert annotations) identifying the entities present in each message. For instance, FIG. 5 depicts an example of a user interface 500, which may be displayed at the client device 130 to receive one or more user inputs identifying the entities present in a message. Alternatively and/or additionally, the labels may be applied automatically such as through keyword matching, regular expressions, or using a machine learning model that accepts message content as input and generates one or more labels as outputs. In some cases, at least some of the automatically determined labels may be verified based on one or more user inputs indicating whether the automatically determined labels accurately identify the corresponding entities. Moreover, in some instances, a single message and a single entity within a message may be associated with multiple labels. The available labels may be specified as a configuration of the system or trained into the system via the machine learning model.

In example embodiments, once the message exchange 110 parses the unstructured clinical data 210 to generate the corresponding structured clinical data 220, the structured clinical data 220 may be passed to the analysis engine 120 for additional downstream tasks. For instance, in the example shown in FIG. 1, the resource controller 125 at the analysis engine 120 may apply, to the structured clinical data 220, one or more machine learning models 170 trained to identify actionable messages as well as extract clinically significant data to enable clinical decisions such as the collection of workflow statistics, resource allocation, and/or the like. In some cases, an actionable message and the clinically significant data contained therein may be determined based on the entities present in the structured clinical data 220. Accordingly, in some cases, the annotated training data used to train the machine learning model 160 may be further associated with one or more ground truth labels indicating whether each message is an actionable message as well as identifying the clinically significant data contained therein. The training of the one or more machine learning models 170 may thus include applying the one or more machine learning models 170 to assess the clinical significance of the messages included in the annotated training data based on sequences of labels, timing of events associated with specific labels, and the like. Trained as such, the output of the one or more machine learning models 170 may enable the analysis engine 120 to efficiently filter messages as received from the data systems 140 and escalate those identified as needing clinical action. In some implementations, when an event having clinical significance is identified, the analysis engine 120 may transmit an alert to one or more devices. In some implementations, the clinical significance may be acute to a specific clinical need. In such implementations, the analysis engine 120 may transmit a control message to one or more clinical devices to cause administration of a clinical response or other workflow (e.g., administration of a drug from an infusion pump, dispensing of a drug from an automated dispensing equipment, performing an assay or other test via a networked diagnostic or analytical device) or cause the clinical device to prepare for a clinical response (e.g., adjust power state, connect to a network, configure operational parameter (e.g., login, assay selection, control variable (e.g., pump rate, dispense location, etc.))).

As used herein, the terms “control” or “controlling” encompass a wide variety of actions. For example, “controlling” a device may include transmitting one or more messages to adjust an operational state or functional element of the device. The message may include specific instructions to be executed by a processor of the device to manifest the change. The “controlling” may include storing a value in a location of a storage device for subsequent retrieval by the device to be controlled, transmitting a value directly to the device to be controlled via at least one wired or wireless communication medium, transmitting or storing a reference to a value, and the like. For example, a control message may include a value to adjust a level of power from a power source of the controlled device. As another example, a control message may activate or deactivate a structural element of the controlled device such as a light, audio playback, a motor, a lock, a pump, a display, or other component of a device described herein. “Controlling” may include indirect control of the device by adjusting a configuration value used by the controlled device. For example, the control message may include a threshold value for a device characteristic (e.g., temperature, rate, frequency, etc.). The threshold value may be stored in a memory location and referred to by the controlled device during operation.

The message exchange 110 may, for example, receive messages from the laboratory information system 145a while the laboratory information system 145a is engaged in a workflow such as microbial testing, virology assay, and/or the like. For the microbial testing workflow, the laboratory information system 145a may generate one or more actionable messages indicating, for example, a start of a culturing process for a microbe, a gram positive or gram negative identification for the microbe, a species and/or organism identification for the microbe, an antimicrobial susceptibility of the microbe, and/or the like. In addition to the one or more actionable messages, the laboratory information system 145a may also generate non-actionable messages transitional status messages and/or the like. The content and/or format of the messages from the laboratory information system 145a may be different from the content and/or format of the messages generated by the other data systems 140, such as the access control system 145b, the dispensing system 145c, the electronic medical record (EMR) system 145d, and/or the like. Absent a uniform data interface between the data systems 140, critical data generated by one data system, such as the laboratory information system 145, may not be available to the other data systems in a timely manner. A lack of interoperability between the data systems 140 may impair the speed, efficiency, and outcome of various clinical workflows that may depend on high throughput interactions or systems that generate high volumes of data (e.g., hundreds or thousands of messages) that may not be processed in an actionable manner without the technical features described.

In some example embodiments, to provide a uniform data interface between the data systems 140, the message exchange 110 may parse the unstructured clinical data 210 included in the messages generated by the laboratory information system 145a by at least applying the machine learning model 160 to identify the entities contained in the messages and populating a data structure (e.g., a vector, a matrix, a table, and/or the like) with the values of the entities extracted from the unstructured clinical data 210 in order to generate the structured clinical data 220. In some cases, the analysis engine 120 may apply, to the structured clinical data 220, the one or more machine learning models 170 to identify actionable messages and extract clinically significant data to enable downstream tasks and decisions. By operating on the structured clinical data 220 instead of the unstructured clinical data 210, the variations in the content and format of messages originating from different medical devices, clinical facilities, and automation platforms may be transparent to the analysis engine 120. For the microbial testing workflow, for example, the one or more machine learning models 170 may be applied to the structured clinical data 220 to identify one or more actionable messages generated by the laboratory information system 145a as well as extract clinically significant data, such as the identification of cultural isolates and the results of antimicrobial susceptibility tests (AST). The clinically significant data extracted from these actionable messages may support further downstream tasks and decisions such as the collection of workflow statistics, resource allocation, and/or the like.

Some messages may be identified as individually actionable. Alternatively and/or additionally, some messages may be considered actionable as part of a group of messages or a sequence of messages. For example, a single message indicating that a specimen encountered at the laboratory information system 145a lacks antibiotic susceptibility may not be actionable on its own (or may constitute one type of an actionable event). Contrastingly, a sequence of messages (e.g., a sequence of more than a threshold quantity of messages) indicating a lack antibiotic susceptibility associated with multiple specimens encountered at the laboratory information system 145a may constitute an actionable event (or a different type of actionable event). Accordingly, the one or more machine learning models 170 may be configured to operate on multiple messages in order to detect actionable events that occur across a group of messages, a sequence of messages, and/or the like. For instance, the one or more machine learning models 170 may include at least one machine learning model trained to operate on sequential data. Examples of such a machine learning model may include a recurrent neural network (RNN), a hidden Markov model, a conditional random field (CRF) model, a gated recurrent unit (GRU), and/or the like.

As noted, clinically significant data may be extracted from actionable messages in order to enable one or more downstream tasks. For example, the analysis engine 120 may identify, based at least on a timestamp associated with the actionable messages, bottlenecks present in the microbial testing workflow in which one or more laboratory activities (e.g., inoculation, incubation, plate imaging, culture reading, result reporting, and/or the like) are associated with an above threshold delay. The analysis engine 120 may determine one or more corrective actions to minimize the bottlenecks including, for example, modifying the scheduling of the laboratories activities, adjusting the allocation of resources associated with the laboratories activities, and/or the like. Alternatively and/or additionally, when the output of the one or more machine learning models 170 indicates the presence of a microbe susceptible to an antimicrobial, for example, in more than a threshold quantity of specimen encountered at the laboratory information system 145a, the resource controller 125 at the analysis engine 120 may determine to allocate a corresponding quantity of the antimicrobial (and/or other resources).

As noted, the one or more machine learning models 170 may be trained to identify actionable messages and extract clinically significant data. In some cases, the one or more machine learning models 170 may be implemented to include a regression model, an instance-based model, a regularization model, a decision tree, a Bayesian model, a clustering model, an associative model, a neural network, a deep learning model, a dimensionality reduction model, an ensemble model, and/or the like. Moreover, in some cases, the one or more machine learning models 170 may include a single machine learning model trained to identify actionable messages as well as extract clinically significant data. For example, the machine learning model may be trained to identify and tag clinically significant data, in which case an actionable message may be a message in which the machine learning model tags more than a threshold quantity of data as clinically significant whereas a non-actionable message may be a message in which the machine learning model does not tag more than a threshold quantity of data as clinically significant. Alternatively, the one or more machine learning engines 170 may include multiple machine learning models such as a first machine learning model trained to identify actionable messages and a second machine learning model trained to extract clinically significant data. Accordingly, messages identified as being actionable by the first machine learning model may be passed to the second machine learning model for the extraction of clinically significant data.

The one or more machine learning models 170 may be trained using training data that includes annotated messages including, for example, messages that have been labeled as actionable or non-actionable, messages that have been labeled with a corresponding stage of the clinical workflow, messages whose content has been tagged to indicate clinically significant data present in the messages, and/or the like. The annotated messages may provide ground truth labels and tags for a supervised learning process in which the one or more machine learning models 170 are trained to identify actionable messages and extract clinically significant data.

In some example embodiments, the one or more machine learning models 170 may be trained to learn an ontology associated with the messages output by the data systems 140 including, for example, the laboratory information system 145a, the access control system 145b, the dispensing system 145c, the electronic medical record (EMR) system 145d, and/or the like. The ontology associated with the messages may define, for example, different categories of messages, the relationship between the different categories of messages, and the data that may be present in each category. By learning the ontology, the one or more machine learning model 170 may be trained to identify messages from different stages of a clinical workflow and to extract the clinically significant data that may arise during each stage of the clinical workflow.

In some example embodiments, the analysis engine 120 may determine, based at least on the output of the one or more machine learning models 170, various workflow statistics. For the microbial testing workflow, for example, the workflow statistics may be determined based at least on a volume and/or a timing of the messages from various stages of the microbial testing workflow (e.g., start of the culturing process for a microbe, a gram positive or gram negative identification for the microbe, a species and/or organism identification for the microbe, an antimicrobial susceptibility of the microbe, and/or the like). Examples of clinical workflow statistics may include various metrics such as a turnaround time (TAT) indicating a distribution of time elapsed between successive stages of a workflow such as the microbial testing workflow associated with the laboratory information system 145a. Alternatively and/or additionally, the analysis engine 120 may identify trends and/or establish benchmarks to enable a comparison between different medical devices, clinical facilities, and automation platforms. These metrics, trends, and/or benchmarks may enable a detection of systematic inefficiencies and bottlenecks across devices, facilities, and automation platforms, which is otherwise unfeasible in the absence of the uniform data interface provided by the message exchange 110.

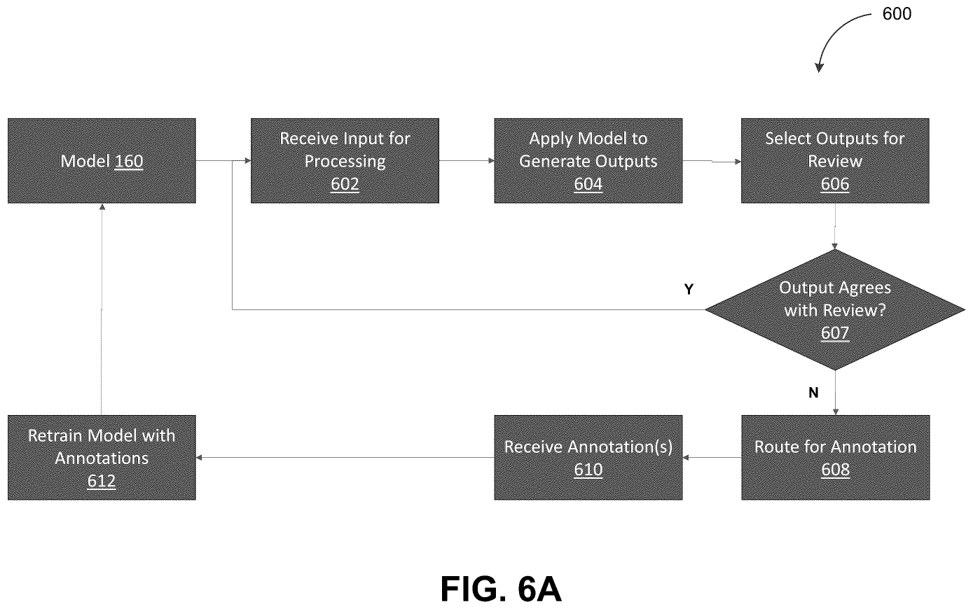

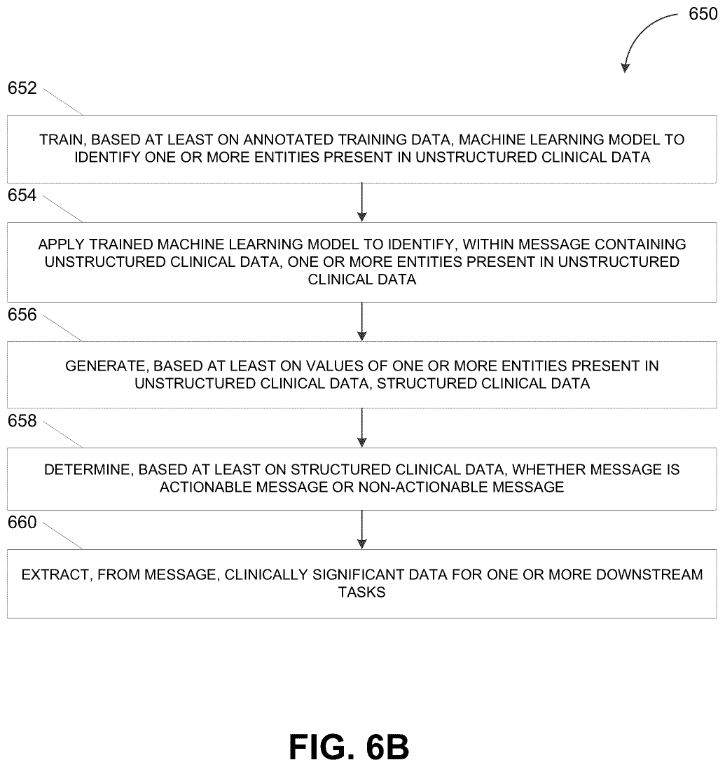

FIG. 6A depicts a flowchart illustrating an example of a process 600 for updating a machine learning model trained to extract unstructured clinical data, in accordance with some example embodiments. Referring to FIGS. 1 and 6A, the process 600 may be performed by the extraction engine 115 of the message exchange 110 in order to update, for example, the machine learning model 160. In some example embodiments, the machine learning model 160 may be trained to parse the unstructured clinical data 210 and generate the corresponding structured clinical data 220. In some cases, the trained machine learning model 160 may undergo subsequent updates in order to maintain and/or improve the performance of the machine learning model 160, for example, when the trained machine learning model 160 encounters data that deviates from the distribution of the training data used to train the machine learning model 160.

At 602, the extraction engine 115 may receive one or more inputs for processing. For example, in some cases, the extraction engine 115 may receive, via the application programming interface 200, one or more requests (e.g., /interpret) containing the unstructured clinical data 210. The unstructured clinical data 210 may form at least a portion of one or more messages originating from the one or more data systems 140 including, for example, the laboratory information system 145a, the access control system 145b, the dispensing system 145c, the electronic medical record system 145d, and/or the like. Some messages may convey clinically significant data and are therefore actionable messages associated with additional tasks while others, such as transitional status messages and/or the like, are non-actionable messages. Nevertheless, the unstructured clinical data 210 contained in the messages may have different format and/or content that vary across different medical devices, clinical facilities, and automation platforms.

At 604, the extraction engine 115 may apply the machine learning model 160 to the inputs to generate one or more outputs. In some example embodiments, the extraction engine 115 may apply the machine learning model 160 to the unstructured clinical data 210 in order to parse the unstructured clinical data 210 and generate the corresponding structured clinical data 220. For example, in some cases, the machine learning model 160 may be trained to identify one or more entities included in the unstructured clinical data 210 including by assigning an entity marker to each entity present in the unstructured clinical data 210. Moreover, in some cases, the machine learning model 160 may generate the structured clinical data 220 by at least populating a data structure (e.g., a vector, a matrix, a table, and/or the like) with the values of the entities extracted from the unstructured clinical data 210.

At 606, the extraction engine 115 may select at least a portion of the one or more outputs of the machine learning model 160 for review. For example, in some cases, the extraction engine 115 may select, from each message, one or more of the entity markers assigned by the machine learning model 160 for further review. Alternatively and/or additionally, the extraction engine 115 may select one or more of the messages processed by the machine learning model 160 and the entity markers assigned to these messages for further review.

At 607, the extraction engine 115 may determine whether the selected outputs of the machine learning model 160 are consistent with the results of the review. For example, in some cases, the extraction engine 115 may determine whether the entity markers assigned by the machine learning model 160 matches the entity markers assigned to the same entities and/or messages by one or more expert annotators. In some cases, the extraction engine 110 may determine whether the quantity of entity markers assigned by the machine learning model 160 that matches those assigned by the expert annotators satisfy one or more thresholds. For example, in some cases, the extraction engine 110 may determine whether more than a threshold quantity or proportion of entity markers assigned by the machine learning model 160 matches those assigned by the expert annotators.

At 607—Y, the extraction engine 115 may determine that the selected outputs of the machine learning model 160 are consistent with the results of the review. Accordingly, the process 600 may resume at operation 602 where the extraction engine 115 continues to receive inputs for processing followed by operation 604 where the extraction engine 115 applies the machine learning model 160 to the inputs to generate outputs. For example, in cases where the quantity of entity markers assigned by the machine learning model 160 that matches those assigned by the expert annotators satisfies the one or more thresholds, the extraction engine 115 may determine to forgo any updating of the machine learning model 160. Instead, the process 600 may resume at operation 602 such that the extraction engine 115 may continue to receive inputs for processing by the machine learning model 160.

Alternatively, at 607—N, the extraction engine 115 may determine that the selected outputs of the machine learning model 160 are not consistent with the results of the review. Accordingly, at 608, the extraction engine 115 may route the inputs for annotation. Moreover, at 610, the extraction engine 115 may receive one or more annotations for the inputs. At 612, the extraction engine 115 may update the machine learning model 160 by at least retraining the machine learning model 160 based on the annotated inputs. In some example embodiments, the trained machine learning model 160 may generate outputs, such as entity markers, that fail to match the results of the review in instances where the inputs provided to the trained machine learning model deviates from the distribution of the training data used to train the machine learning model 160. In cases where the quantity of entity markers assigned by the machine learning model 160 that matches those assigned by the expert annotators fails to satisfy the one or more thresholds, the extraction engine 115 may determine to update the machine learning model 160. For example, the extraction engine 115 may route the inputs provided to the machine learning model 160 in operation 604 for expert annotation and/or automatic annotation. Furthermore, the annotated inputs may be used as training data to retrain the machine learning model 160. Doing so may improve the performance of the machine learning model 160, for example, by increasing the accuracy of the entity markers assigned by the machine learning model 160.

FIG. 6B depicts a flowchart illustrating an example of a process 650 for machine learning enabled extraction of unstructured clinical data, in accordance with some example embodiments. Referring to FIGS. 1 and 6B, the process 650 may be performed by the message exchange 110, for example, the extraction engine 115, in order to train and apply the machine learning model 160 to extract the unstructured clinical data 210 that may be present in the messages generated by the one or more data systems 140. Moreover, in some cases, the process 650 may be performed by the analysis engine 120 to identify clinical actionable messages and extract clinically significant data for downstream tasks.

At 652, the extraction engine 115 may train, based at least on annotated training data, the machine learning model 160 to identify one or more entities present in unstructured clinical data. In some example embodiments, the machine learning model 160 may be trained to learn various entity patterns that may be present in the unstructured clinical data included in the messages generated by the one or more data systems 140. For example, in some cases, the machine learning model 160 may be trained based on the entity pattern catalog 350, which may include unstructured clinical data that have been annotated with entity markers identifying the various entities contained therein. In some case, the machine learning model 160 may be a conditional random fields (CRF) model that is trained to learn sequences of entities that form, for example, vertical chains as well as horizontal chains in unstructured clinical data. That is, in some cases, the machine learning model 160 may be trained to learn entity patterns in which two or more entities appear in a specific order in a same row or across successive rows of the unstructured clinical data. Accordingly, when trained, the machine learning model 160 may, upon identifying the first entity in the unstructured clinical data, determine that the second entity is the most likely next entity in the same row of the unstructured clinical data or in a next row in the same column of the unstructured clinical data.

At 654, the extraction engine 115 may apply the trained machine learning model 160 to identify, in a message containing unstructured clinical data, one or more entities present in the unstructured clinical data. In some example embodiments, the extraction engine 115 may apply the trained machine learning model 160 to identify one or more entities present in the unstructured clinical data 210 included in the messages received from the one or more data systems 140. In some cases, the trained machine learning model 160, which has been trained to learn various entity patterns that may be present in the unstructured clinical data 210, may identify each entity present in the unstructured clinical data 210 based on the other entities that appear before (or after) the entity, for example, in a same row or in different row in a same column of the unstructured clinical data 210. Moreover, in some cases, the trained machine learning model 160 may identify each entity by at least assigning a corresponding entity marker to the entity.

At 656, the extraction engine 115 may generate, based at least on the values of the one or more entities present in the unstructured clinical data, structured clinical data. In some example embodiments, upon identifying the entities that are present in the unstructured clinical data 210, the extraction engine 115 may generate the corresponding structured clinical data 220 by at least populating a data structure (e.g., a vector, a matrix, a table, and/or the like) with the values of the entities extracted from the unstructured clinical data 210.

At 658, the analysis engine 120 may determine, based at least on the structured clinical data, whether the message is an actionable message or a non-actionable message. In some example embodiments, the message controller 110 may send, to the analysis engine 120, the structured clinical data 220 for one or more downstream tasks. For example, for a microbial testing workflow, the analysis engine 120 may apply the one or more machine learning model 170 determine that a message from the laboratory information system 145a is associated with the start of the culturing process for a microbe, a gram positive or gram negative identification for the microbe, a species and/or organism identification for the microbe, an antimicrobial susceptibility of the microbe, and/or the like.

At 660, the analysis engine 120 may extract, from the message, clinically significant data for one or more downstream tasks. For example, in some cases, the analysis engine 120 may apply the one or more machine learning models 170 to extract, from the messages generated by the laboratory information system 145a, clinically significant data such as the identification of cultural isolates and the results of antimicrobial susceptibility tests (AST). In some example embodiments, clinically significant data extracted from one or more actionable messages may enable one or more downstream tasks and/or decisions. Examples of downstream tasks and/or decisions, which may be performed at the analysis engine 120, may include the collection of workflow statistics, resource allocation, and/or the like.

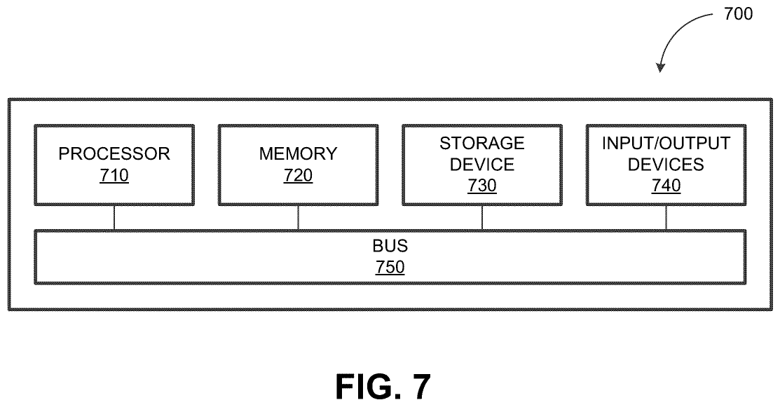

FIG. 7 depicts a block diagram illustrating a computing system 700 consistent with implementations of the current subject matter. Referring to FIGS. 1 and 7, the computing system 700 can be used to implement the message exchange 110, the analysis engine 120, and/or any components therein.

As shown in FIG. 7, the computing system 700 can include a processor 710, a memory 720, a storage device 730, and input/output device 740. The processor 710, the memory 720, the storage device 730, and the input/output device 740 can be interconnected via a system bus 750. The processor 710 is capable of processing instructions for execution within the computing system 700. Such executed instructions can implement one or more components of, for example, the message exchange 110 and/or the analysis engine 120. In some example embodiments, the processor 710 can be a single-threaded processor. Alternatively, the processor 710 can be a multi-threaded processor. The processor 710 is capable of processing instructions stored in the memory 720 and/or on the storage device 730 to display graphical information for a user interface provided via the input/output device 740.

The memory 720 is a computer readable medium such as volatile or non-volatile that stores information within the computing system 700. The memory 720 can store data structures representing configuration object databases, for example. The storage device 730 is capable of providing persistent storage for the computing system 700. The storage device 730 can be a floppy disk device, a hard disk device, an optical disk device, a tape device, a solid-state device, and/or any other suitable persistent storage means. The input/output device 740 provides input/output operations for the computing system 700. In some example embodiments, the input/output device 740 includes a keyboard and/or pointing device. In various implementations, the input/output device 740 includes a display unit for displaying graphical user interfaces.

According to some example embodiments, the input/output device 740 can provide input/output operations for a network device. For example, the input/output device 740 can include Ethernet ports or other networking ports to communicate with one or more wired and/or wireless networks (e.g., a local area network (LAN), a wide area network (WAN), the Internet).

In some example embodiments, the computing system 700 can be used to execute various interactive computer software applications that can be used for organization, analysis and/or storage of data in various formats. Alternatively, the computing system 700 can be used to execute specific unstructured clinical data software applications. These applications can be used to perform various functionalities, e.g., planning functionalities (e.g., generating, managing, editing of spreadsheet documents, word processing documents, and/or any other objects, etc.), computing functionalities, communications functionalities, etc. related to the unstructured clinical data processing features described. The applications can include various add-in functionalities or can be standalone computing products and/or functionalities. Upon activation within the applications, the functionalities can be used to generate the user interface provided via the input/output device 740. The user interface can be generated and presented to a user by the computing system 700 (e.g., on a computer screen monitor, etc.).

One or more aspects or features of the subject matter described herein can be realized in specifically configured digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a specifically configured computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data can be forwarded to a “remote” device or location,” where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

One or more aspects of the artificial intelligence described may be implemented in whole or in part by a model. A model may be implemented as a machine learning model. The learning may be supervised, unsupervised, reinforced, or a hybrid learning whereby multiple learning techniques are employed to generate the model. The learning may be performed as part of training. Training the model may include obtaining a set of training data and adjusting characteristics of the model to obtain a desired model output. For example, three characteristics may be associated with a desired device state. In such instance, the training may include receiving the three characteristics as inputs to the model and adjusting the characteristics of the model such that for each set of three characteristics, the output device state matches the desired device state associated with the historical data.

In some implementations, the training may be dynamic. For example, the system may update the model using a set of events. The detectable properties from the events may be used to adjust the model.

The model may be an equation, artificial neural network, recurrent neural network, convolutional neural network, decision tree, or other machine readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds or limits for evaluating an output value, or criterion for selecting from a set of equations.

Once a model is trained, retraining may be included to refine or update the model to reflect additional data or specific operational conditions. The retraining may be based on one or more signals detected by a device described herein or as part of a method described herein. Upon detection of the designated signals, the system may activate a training process to adjust the model as described.

Further examples of machine learning and modeling features which may be included in the embodiments discussed above are described in “A survey of machine learning for big data processing” by Qiu et al. in EURASIP Journal on Advances in Signal Processing (2016) which is hereby incorporated by reference in its entirety.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device, diagnostic device, monitoring device, or server in communication therewith.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A computer-implemented method, comprising:
receiving, from one or more data systems, a message including unstructured clinical data;
applying a machine learning model to identify a first entity and a second entity present in the unstructured clinical data, the first entity and the second entity occupying a same row of the unstructured clinical data or successive rows in a same column of the unstructured clinical data, the machine learning model being trained to determine, based at least on the unstructured clinical data including the first entity, that the second entity is a most likely entity occupying a next position in the same row or a next row in the same column of the unstructured clinical data;
extracting, from the structured clinical data, a clinically significant data; and
controlling, based at least on the clinically significant data, at least one medical device to perform one or more tasks.

2. The method of claim 1, wherein the machine learning model is trained to identify the second entity included in the unstructured clinical data by at least:
determining, based at least on the unstructured clinical data including the first entity, a first probability that the second entity is the most likely entity occupying the next position in the same row or the next row in the same column of the unstructured clinical data,
determining, based at least on the unstructured clinical data including the first entity, a second probability that a third entity is the most likely entity occupying the next position in the same row or the next row in the same column of the unstructured clinical data,
identifying the second entity based at least on a comparison of the first probability and the second probability.

3. The method of claim 1, further comprising:
generating, based at least on an alignment between the first entity and the second entity in the unstructured clinical data, an entity graph including the first entity and the second entity.

4. The method of claim 3, further comprising:
generating, based at least on a horizontal alignment between the first entity and the second entity, a horizontal chain of the entity graph to include the first entity and the second entity such that the first entity and the second entity occupy a single row.

5. The method of claim 3, further comprising:
generating, based at least on a vertical alignment between the first entity and the second entity, a vertical chain of the entity graph to include the first entity and the second entity such that the first entity and the second entity occupy successive rows in a single column.

6. The method of claim 1, wherein the machine learning model identifies the first entity and the second entity by at least assigning, to each of the first entity and the second entity, a corresponding entity marker.

7. The method of claim 1, wherein the at least one medical device is controlled based on at least one of a header, an organism name, a drug name, a concentration value, and an interpretation value comprising the first entity and/or the second entity.

8. The method of claim 1, further comprising:
training, based at least on training data including annotated unstructured clinical data, the machine learning model to learn one or more entity patterns that are present in the annotated unstructured clinical data, the annotated unstructured clinical data including a plurality of entities annotated with a corresponding ground truth entity marker.

9. The method of claim 8, wherein the one or more entity patterns include a co-occurrence of the first entity and the second entity in a single row or in successive rows of a single column.

10. The method of claim 1, wherein the one or more tasks include:
identifying, based at least on the clinically significant data, a stage of a clinical workflow associated with the one or more messages,
determining, based at least on a timestamp associated with the one or more messages, a quantity of time between two or more successive stages of the clinical workflow, and
in response to the quantity of time between the two or more successive stages of the clinical workflow exceeding a threshold value, determining one or more corrective configurations for the at least one medical device.

11. The method of claim 10, wherein the one or more corrective actions include modifying a scheduling of one or more activities associated with the clinical workflow and/or adjusting an allocation of resources associated with the one or more activities.

12. The method of claim 10, wherein the identifying of the stage of the clinical workflow comprises identifying the stage of a microbial testing workflow and/or a virology assay.

13. The method of claim 10, wherein the identifying the stage of the clinical workflow comprises identifying a start of a culturing process for a microbe, a gram positive or gram negative identification for the microbe, a species and/or organism identification for the microbe, or an antimicrobial susceptibility of the microbe.

14. The method of claim 10, wherein the one or more tasks include determining, based at least on the clinically significant data, an allocation of resources at the one or more data systems.

15. The method of claim 14, wherein the allocation of resources includes:
determining, based at least on the clinically significant data, a subsequent stage of a clinical workflow and a time for the subsequent stage of the clinical workflow, and
scheduling, in accordance with the time of the subsequent stage of the clinical workflow, a quantity of resources required for the subsequent stage of the clinical workflow.

16. The method of claim 1, further comprising:
applying one or more second machine learning models to extract, from the structured clinical data, the clinically significant data.

17. The method of claim 16, wherein the one or more second machine learning models are trained to identify and tag the clinically significant data included in the structured clinical data, and wherein the one or more second machine learning models are further trained to determine that the message associated with the structured clinical data is actionable in response to more than a threshold portion of the structured clinical data being tagged as clinically significant.

18. The method of claim 17, wherein the one or more second machine learning models are further trained to determine, based at least on a sequence of messages including the message, whether the sequence of messages is actionable.

19. The method of claim 1, wherein the controlling of the at least one medical device includes transmitting, to the at least one medical device, one or more messages to adjust an operational state and/or a functional element of the at least one medical device.

20. The method of claim 19, wherein the one or more messages include one or more instructions, which when executed by a processor associated with the at least one medical device, adjust the operational state and/or the functional element of the at least one medical device.

21. The method of claim 19, wherein the one or more messages include one or more values, which when applied at the at least one medical device, adjust the operational state and/or the functional element of the at least one medical device.

22. The method of claim 19, wherein the at least one medical device comprises an infusion pump including a pumping functional element, and wherein the method comprises generating the one or more messages to adjust a pumping rate of the pumping functional element.

23. The method of claim 19, wherein the at least one medical device comprises a dispensing cabinet or a wasting station including one or more receptacles, and wherein the method comprises generating the one or more messages to control access to the one or more receptacles.

24. A system, comprising:
at least one data processor; and
at least one memory storing instructions, which when executed by the at least one data processor, result in operations comprising the method of claim 1.

25. A non-transitory computer readable medium storing instructions, which when executed by at least one data processor, result in operations comprising the method of claim 1.
